(19) United States
(12) Patent Application Publication
(10) Pub. No.: US 2026/0183466 A1
(43) Pub. Date: Jul. 2, 2026

(54) FLUID REGULATING APPARATUS WITH INNER AND OUTER SUCTION LUMENS

(71) Applicant: ResQ Medical Ltd., Yokneam (IL)

(72) Inventors: Itamar Dach, Pardes Hanna (IL); Amnon Weichselbaum, Haifa (IL); Tal Youdim, Kiryat Tivon (IL)

(73) Assignee: ResQ Medical Ltd., Yokneam (IL)

(21) Appl. No.: 19/001,595

(22) Filed: Dec. 26, 2024

(51) Int. Cl. A61M 1/00

(52) U.S. Cl. CPC A61M 1/7415; A61M 2202/0014; A61M 2205/054; A61M 2205/3344

(57) ABSTRACT

A body fluid regulating apparatus includes an inner tube disposed inside an outer tube with a gap between the inner and outer tubes. The inner tube and outer tubes are each formed with apertures that are open to the gap. A first suction lumen is located in the inner tube adapted to be coupled to a vacuum source for creating a negative pressure in the inner tube. A second suction lumen is adapted to be coupled to the vacuum source or to another vacuum source for creating a negative pressure in the apertures of the outer tube. A plug is disposed on a proximal portion the outer tube distant from the apertures of the inner and outer tubes.

### FIELD OF THE INVENTION

The present invention relates to a body fluid regulating apparatus, such as for treating postpartum hemorrhage, which has inner and outer suction lumens.

### BACKGROUND OF THE INVENTION

PCT Patent application WO 2024/127158 describes a body fluid regulating apparatus, particularly a uterine hemorrhage regulating apparatus for treating postpartum hemorrhage, although the apparatus can be used to regulate or stop other body fluids from other organs, such as the urinary bladder and other organs.

The apparatus includes means for seizing, grasping, and holding the uterine cavity tissue (endometrium and or myometrium) while applying negative pressure (vacuum) to the cavity, in order to evacuate blood clots and other debris from the uterus, and stop the bleeding by contracting the uterus and forcing contact between the walls of the uterus. Additional features include, for example, injection of various fluids or other substances into the uterus cavity or directly into the endometrium/myometrium, such as substances that enhance uterine contractility

### SUMMARY

The present invention seeks to provide a body fluid regulating apparatus, such as for treating postpartum hemorrhage, with advanced features that include inner and outer suction lumens. Although the invention is described with reference to a uterine hemorrhage regulating apparatus, the invention can be used to regulate or stop other body fluids gases, gels, semifluid from other organs, such as the urinary bladder and other organs.

In the present invention, not only is the inner tube an active suction tube (that is, a suction lumen is used to create suction through the apertures of the inner tube into the inner tube), the outer tube is also an active suction tube because there is another suction lumen which is used to create suction through the apertures of the outer tube into the outer tube. This other suction lumen may be located in the gap between the inner and outer tubes, or the outer tube may be a sheath placed over the inner tube and the other suction lumen may be formed in the sheath, as described below. Both tubes may be used to suck tissue towards the tube, such as to stop hemorrhaging. The use of two active suction tubes provides effective suction on the tissue without any shielding of the tubes and reduces the risk of tube clogging.

Elongated ribs may be provided to maintain a gap between the outer tube and the inner tube and prevent the outer tube from collapsing against the inner tube.

The apparatus may include an anchoring inflatable plug or seal, such as a balloon. The inflatable plug, which may be fixed or slidable and adjustable, may be placed in the cervix or vagina. When expanded, the plug blocks the opening of the uterus, and thereby fixes the apparatus in place and allows a vacuum to be created in the uterine cavity.

The vacuum source (such as a vacuum pump) is coupled to the inner and outer tubes to create negative pressure that sucks the uterine walls towards the tubes. The uterine walls press against and around the outer tube (portions of the uterine walls press against each other to help stop the bleeding), but do not press or abut against the inner tube. Vacuum in both inner and outer suction lumens is maintained.

There is provided in accordance with a non-limiting embodiment of the present invention body fluid regulating apparatus including an inner tube disposed inside an outer tube with a gap between the inner and outer tubes, the inner tube and the outer tube each being formed with apertures that are open to the gap, a first suction lumen located in the inner tube adapted to be coupled to a vacuum source for creating a negative pressure in the inner tube, a second suction lumen adapted to be coupled to the vacuum source or to another vacuum source for creating a negative pressure in the apertures of the outer tube, and an inflatable plug disposed on a proximal portion the outer tube distant from the apertures of the inner and outer tubes.

In accordance with a non-limiting embodiment of the present invention the second suction lumen is located in the gap.

In accordance with another non-limiting embodiment of the present invention the outer tube includes a sheath disposed over a distal portion of the inner tube, and the second suction lumen is located in the sheath.

In accordance with a non-limiting embodiment of the present invention the outer tube includes a deformable portion which can be deformed to regulate an amount of suction in the second suction lumen.

In accordance with a non-limiting embodiment of the present invention the sheath is formed with spiral protrusions with vacuum ports located between the spiral protrusions.

In accordance with a non-limiting embodiment of the present invention a negative pressure regulator is coupled to the first and second suction lumens, the negative pressure regulator being set to select which of the first and second suction lumens has suction being drawn therethrough, and to regulate an amount of suction in each of the first and second suction lumens.

In accordance with a non-limiting embodiment of the present invention a needle passes through the sheath.

In accordance with a non-limiting embodiment of the present invention needle includes a grasping member.

In accordance with a non-limiting embodiment of the present invention the needle includes a stimulating electrode operative to stimulate muscle contraction by means of electrical impulses.

In accordance with a non-limiting embodiment of the present invention one or more elongated ribs extend between the inner and outer tubes, the one or more elongated ribs being coupled to at least one of the inner and outer tubes.

In accordance with a non-limiting embodiment of the present invention the sheath includes a drug-eluting sheath for delivering a drug to a tissue.

### BRIEF DESCRIPTION OF DRAWINGS

The present invention will be understood and appreciated more fully from the following detailed description, taken in conjunction with the drawings in which:

FIG. 1 is a simplified illustration of a body fluid regulating apparatus, in accordance with a non-limiting embodiment of the present invention, including inner and outer tubes.

FIG. 2 is a simplified illustration of a proximal portion of the body fluid regulating apparatus, showing the inner and outer vacuum (suction) lumens.

FIG. 3 is an end view of the body fluid regulating apparatus, showing the inner and outer vacuum (suction) lumens.

FIG. 4 is a simplified illustration of a flexible portion of the outer tube which can be pressed (deformed) to regulate the amount of suction in the outer suction lumen, in accordance with a non-limiting embodiment of the present invention.

FIGS. 5A and 5B are simplified side-view and end-view illustrations, respectively, of a sheath for the body fluid regulating apparatus, in accordance with another non-limiting embodiment of the present invention, the sheath being formed with spiral protrusions with vacuum ports located between the spirals.

FIG. 6 is a simplified illustration of a negative pressure regulator for use with the body fluid regulating apparatus, which can be used to select which suction lumen is in use and the level of suction in each suction lumen.

FIG. 7 is a simplified illustration of a sheath coupled to the distal tip of the body fluid regulating apparatus, in which the outer suction lumen passes through the sheath.

FIG. 8 is a simplified illustration of a needle passing through a sheath of the body fluid regulating apparatus, in accordance with another non-limiting embodiment of the present invention.

### DETAILED DESCRIPTION

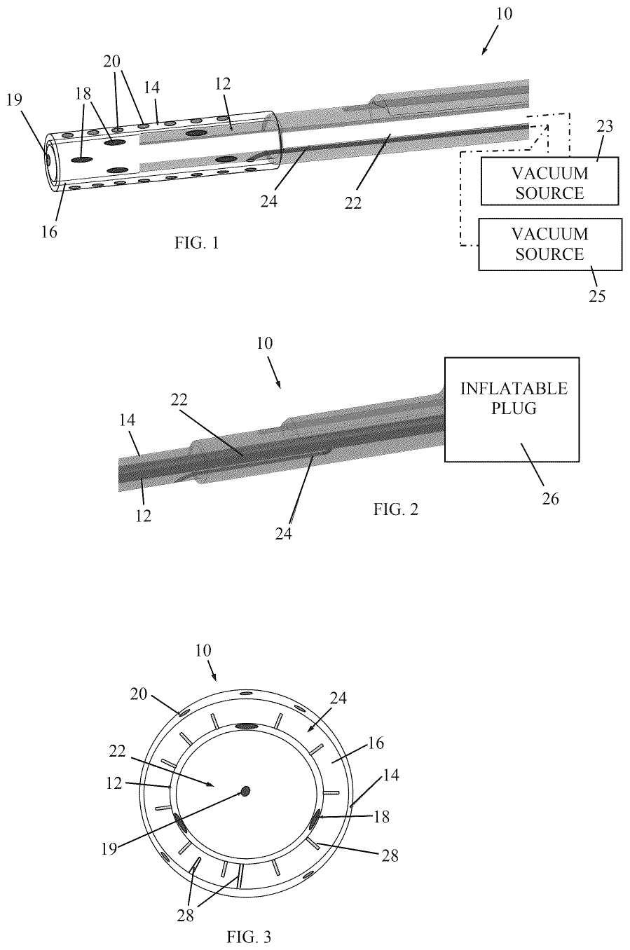

Reference is now made to FIGS. 1-3, which illustrate a body fluid regulating apparatus 10, in accordance with a non-limiting embodiment of the present invention.

The apparatus 10 may include an inner tube 12 inside an outer tube 14 (the two tubes together are also called the intrauterine shaft or simply shaft) with a gap 16 between the two tubes. The apparatus 10 may include a proximal handle (not shown, but may be similar to that shown in PCT Patent application WO 2024/127158).

Gap 16 and the lumen of the internal tube 12 may be additionally used as a working channel for tools, such as but not limited to, fiber optic tools, hysteroscopy tools, graspers, biopsy tools and more.

The inner tube 12 has a smaller diameter than the outer tube 14 and is formed with apertures 18 which may or may not be larger in size than apertures 20 formed in the outer tube 14. The apertures 18 and 20 may be of any size and shape, such as but not limited to, circular, elliptical, polygonal, regular, irregular, etc. For example, without limitation, apertures 18 may be oval in the range of 2-6 mm in width and 5-10 mm in length; apertures 20 may be oval in the range of 1-5 mm in width and 3-7 mm in length The apertures 18 and 20 may be formed in any pattern around the circumference, and along the length, of inner tube 12 and outer tube 14, respectively.

As seen in FIGS. 1 and 3, another aperture 19 may be formed at the distal end of inner tube 12.

A first suction lumen 22 is located in inner tube 12 and is adapted to be coupled to a vacuum source 23 (FIG. 1) for creating a negative pressure in inner tube 12. A second suction lumen 24 is adapted to be coupled to vacuum source 23, or to another vacuum source 25, for creating a negative pressure in apertures 20 of outer tube 14.

It is noted that in the embodiment of FIGS. 1-3, the second suction lumen 24 is located in gap 16. Optionally, outer tube 14 may be a sheath, that is, a mesh or porous covering or other kinds of covering with one or more apertures and the term “outer tube” encompasses all of these structures. As seen later in FIG. 7, in such a case, the second suction lumen may be in the sheath.

Reference is now made to FIG. 2, which illustrates the proximal portion of the body fluid regulating apparatus 10. Apparatus 10 may include an anchoring inflatable plug 28, such as a balloon. The inflatable plug 26 may be slidable along outer tube 14. When expanded, inflatable plug 26 blocks the opening of the uterus, and thereby fixes apparatus 10 in place and allows a vacuum to be created in the uterine cavity. Alternatively, the plug can be a solid, non-inflatable plug.

Referring to FIG. 3, it is seen that one or more elongated ribs 28 may extend between inner and outer tubes 12 and 14. The ribs 28 may be coupled to inner tube 12, or to outer tube 14 or to both tubes 12 and 14. Ribs 28 are positioned between the apertures so they do not interfere with the apertures. Ribs 28 maintain a gap between the outer tube 14 and the inner tube 12 and prevent the outer tube 14 from collapsing against the inner tube 12.

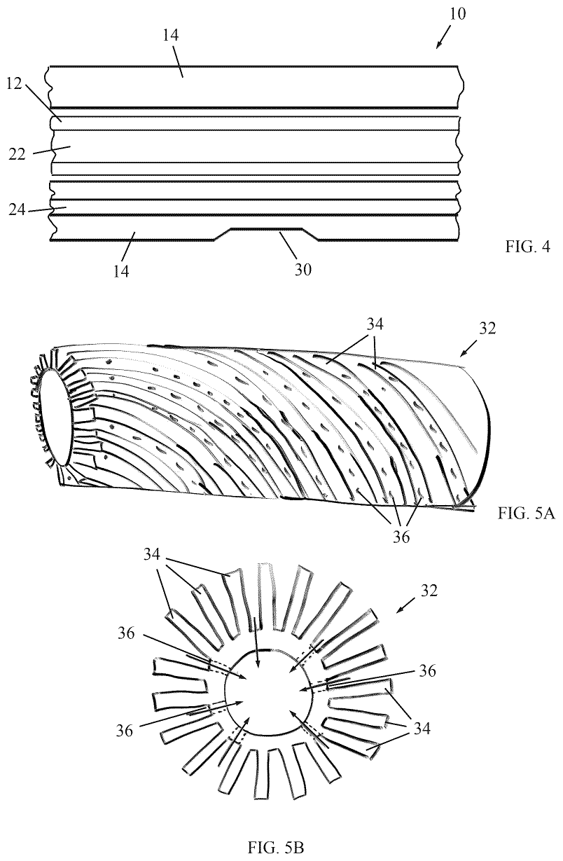

Reference is now made to FIG. 4, which illustrates another feature that is possible with the invention. The outer tube 14 may include a deformable portion 30, such as a thinner portion and/or a portion made of soft or flexible material, which can be deformed (such as by the user pressing it with a finger) to regulate an amount of suction in second suction lumen 24. Pressing in the deformable portion 30 decreases the pathway for the suction, thereby reducing the amount of suction. This mechanism enables the user to precisely control the suction level, facilitating adjustable fluid evacuation and aiding in uterine contraction management.

Reference is now made to FIGS. 5A and 5B, which illustrate a sheath 32 for the body fluid regulating apparatus, in accordance with another non-limiting embodiment of the present invention. The sheath 32 may be formed with spiral (also referred to as helical) protrusions 34 with vacuum ports 36 located between the spiral protrusions 34. This structure may enhance the suction power of the apparatus and may increase the suction of the tissue towards the apparatus.

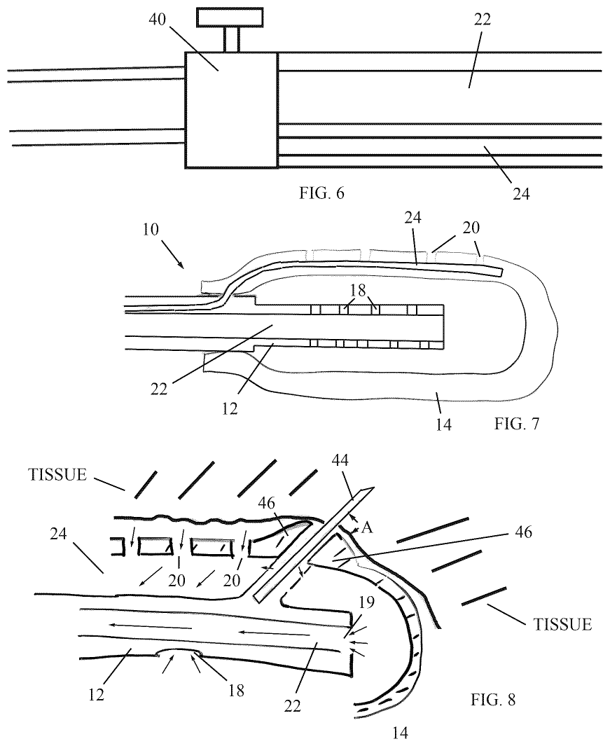

Reference is now made to FIG. 6, which illustrates a negative pressure regulator 40 for use with the body fluid regulating apparatus. The negative pressure regulator 40 may include a manual valve or a valve which works with feedback control from sensors, or based on a timer and other digital and or mechanical mechanisms and is in fluid communication with the vacuum source(s) and with the suction lumens, The negative pressure regulator 40 can be used to select which suction lumen is in use and the level of suction in each suction lumen. For example, the negative pressure regulator 40 can be set so that only the first suction lumen 22 provides suction to the treated area, and no suction is provided from the second suction lumen 24. Conversely, negative pressure regulator 40 can be set so that only the second suction lumen 24 provides suction to the treated area, and no suction is provided from the first suction lumen 22. Alternatively, negative pressure regulator 40 can be set so that both the first suction lumen 22 and the second suction lumen 24 provide suction to the treated area. Furthermore, negative pressure regulator 40 can independently adjust the amount of suction in each suction lumen.

Reference is now made to FIG. 7. As stated above, outer tube 14 may be a sheath, In this embodiment, the sheath (outer tube 14) is coupled to the distal tip of the body fluid regulating apparatus 10 and the outer (second) suction lumen 24 passes into the thickness of the sheath to be in fluid communication with apertures 20 of the outer sheath 14, without piercing outwards through the thickness of the sheath.

Reference is now made to FIG. 8, which illustrates that the body fluid regulating apparatus may include one or more needles 44 that pass through the outer tube 14 (e.g., sheath). As similarly described in PCT Patent application WO 2024/127158, after inserting the apparatus into the uterus, inflating the anchoring balloon in the vagina, turning on the vacuum and collapsing the uterine tissue around the tubes, the needles 44 may serve as grasping members which become embedded in the adjacent uterine tissue (mechanical grasping). In addition, drugs for enhancing uterine contractility can be delivered via the needles (grasping members), in which case the needles have a hollow lumen through which a drug can be injected. Additionally or alternatively, the sheath (outer tube 14) may be a drug-eluting sheath for delivering a drug to the uterine tissue. Without limitation, the drug may be a uterotonic agent, oxytocin, or drugs used to enhance uterine contractility, such as but not limited to, ergot alkaloids and prostaglandins, such as metheargine, carboprost and dinoprostone and misoprostol, antibiotics, chemotherapy.

Additionally or alternatively, needles 44 may be stimulating electrodes operative to stimulate muscle contraction by means of electrical impulses (electrical muscle stimulation or electromyostimulation), cryotherapy, heat. The electrodes may be positioned to come into contact with the organ tissue after the negative pressure (vacuum) is operated. The electrodes may be powered by a battery integrated in the device and activated when the procedure begins.

As seen in FIG. 8, needle 44 may protrude through a thickened and/or raised portion 46 of outer tube (sheath) 14. This thickened and/or raised portion 46 helps maintain needle 44 at a desired angular orientation (indicated by angle A in FIG. 8), such as but not limited to, 90° or angles less than 90°, depending on the penetration desired. Without limitation, the needle 44 may be a standard round needle that passes through an oval-shaped hole, ensuring unobstructed passage of vacuum in the space between the oval tube and round needle, ensuring effective penetration and creating space for the vacuum to secure the tissue onto the needle.

1. A body fluid regulating apparatus comprising:
an inner tube disposed inside an outer tube with a gap between said inner and outer tubes, said inner tube and said outer tube each being formed with apertures that are open to said gap;
a first suction lumen located in said inner tube adapted to be coupled to a vacuum source for creating a negative pressure in said inner tube;
a second suction lumen adapted to be coupled to said vacuum source or to another vacuum source for creating a negative pressure in said apertures of said outer tube; and
a plug disposed on a proximal portion said outer tube distant from said apertures of said inner and outer tubes.

2. The body fluid regulating apparatus according to claim 1, wherein said second suction lumen is located in said gap.

3. The body fluid regulating apparatus according to claim 1, wherein said outer tube comprises a sheath disposed over a distal portion of said inner tube, and said second suction lumen is located in said sheath.

4. The body fluid regulating apparatus according to claim 1, wherein said outer tube comprises a deformable portion which can be deformed to regulate an amount of suction in said second suction lumen.

5. The body fluid regulating apparatus according to claim 3, wherein said sheath is formed with spiral protrusions with vacuum ports located between said spiral protrusions.

6. The body fluid regulating apparatus according to claim 1, further comprising a negative pressure regulator coupled to said first and second suction lumens, said negative pressure regulator being set to select which of said first and second suction lumens has suction being drawn therethrough, and to regulate an amount of suction in each of said first and second suction lumens.

7. The body fluid regulating apparatus according to claim 3, wherein a needle passes through said sheath.

8. The body fluid regulating apparatus according to claim 7, wherein said needle comprises a grasping member.

9. The body fluid regulating apparatus according to claim 7, wherein said needle comprises a stimulating electrode operative to stimulate muscle contraction by means of electrical impulses.

10. The body fluid regulating apparatus according to claim 1, further comprising one or more elongated ribs that extend between said inner and outer tubes, said one or more elongated ribs being coupled to at least one of said inner and outer tubes.

11. The body fluid regulating apparatus according to claim 3, wherein said sheath comprises a drug-eluting sheath for delivering a drug to a tissue.
